(19) United States
(12) Patent Application Publication
(10) Pub. No.: US 2019/0218294 A1
(43) Pub. Date: Jul. 18, 2019

(54) USE OF AN ANTI-PD-1 ANTIBODY IN COMBINATION WITH AN ANTI-MESOTHELIN ANTIBODY IN CANCER TREATMENT

(71) Applicant: BRISTOL-MYERS SQUIBB COMPANY, Princeton, NJ (US)

(72) Inventors: Shivani SRIVASTAVA, Princeton, NJ (US); Deanne LATHERS, Doylestown, PA (US); Heather E. VEZINA, Upper Holland, PA (US); Josephine M. CARDARELLI, San Carlos, CA (US); Peter SABBATINI, Trappe, PA (US); Mark S. WADE, Yardley, PA (US); Chin PAN, Los Altos, CA (US)

(21) Appl. No.: 16/331,266

(22) PCT Filed: Sep. 7, 2017
(86) PCT No.: PCT/US2017/050390

§ 371 Date: Mar. 7, 2019

Related U.S. Application Data
(60) Provisional application No. 62/385,597, filed on Sep. 9, 2016.

(51) Int. Cl. C07K 16/28; A61K 47/68; A61P 35/00; C07K 16/30; A61K 31/454

(52) U.S. Cl. CPC C07K 16/2818; A61K 47/6803; A61P 35/00; A61K 47/6851; C07K 16/30; A61K 31/454; A61K 47/6849; C07K 16/2839

(57) ABSTRACT

A method of treating a tumor in a patient by administering to the patient a therapeutically effective amount of a combination of an anti-PD-1 antibody and an anti-mesothelin antibody-drug conjugate.

### CROSS-REFERENCE TO RELATED APPLICATIONS

This application claims the benefit under 35 U.S.C. § 119(e) of U.S. Provisional Application Ser. No. 62/385,597, filed Sep. 9, 2016; the disclosure of which is incorporated herein by reference.

### BACKGROUND OF THE INVENTION

### 1. Technical Field of the Invention

This invention relates to methods for treating a cancer in a subject comprising administering to the subject an anti-Programmed Death-1 (PD-1) antibody and antibody-drug conjugate of an anti-mesothelin antibody.

### 2. Description of Related Art

Patients with metastatic or refractory solid cancers have very poor prognosis. Despite advances in multimodal therapy, increases in overall survival in this patient population have been limited. This unmet need for treatments that deliver long-term survival indicates a need for new treatments that bring to bear novel mechanisms of action or combinations of different modes of action.

Programmed cell death protein 1 (PD-1) is a member of the CD28 family of T cell costimulatory receptors that also includes CD28, cytotoxic T-lymphocyte associated antigen 4 (CTLA-4), ICOS, and BTLA. PD-1 signaling has been shown to inhibit CD-28-mediated upregulation of interleukins 2, 10, and 13, interferon-γ (IFN-γ), and Bcl-xL. PD-1 expression also has been noted to inhibit T cell activation and expansion of previously activated cells. Evidence for a negative regulatory role of PD-1 comes from studies of PD-1-deficient mice, which develop a variety of autoimmune phenotypes. These results suggest that PD-1 blockade has the potential to activate anti-self T cell responses, but these responses are variable and dependent upon various host genetic factors. Thus, PD-1 deficiency or inhibition is not accompanied by a universal loss of tolerance to self-antigens.

Nivolumab (OPDIVO®) is an anti-PD1 antibody that binds to PD-1 in vitro with high affinity and inhibits the binding of PD-1 to its ligands programmed death ligands 1 (PD-L1) and 2 (PD-L2). Nivolumab binds specifically to PD-1 and not to related members of the CD28 family, such as CD28, ICOS, CTLA-4, and BTLA. Nivolumab has been approved for the treatment of certain types of cancer in certain patient populations. Pembrolizumab (KEYTRUDA®) is another anti-PD-1 antibody, which has also been approved for the treatment of certain cancers in certain patient populations. The PD-1 pathway and the mechanism of action of nivolumab are shown in FIG. 1.

Antibodies such as nivolumab and pembrolizumab represent one mechanism of action for anti-cancer treatments. They are immune-oncology agents, that is, they activate the patient's immune system to attack the cancer.

Antibody-drug conjugates (ADCs, sometimes referred to as an immunoconjugate), represent another anti-cancer mechanism of action. In an ADC, a linker covalently links a drug (also referred to as a therapeutic agent, cytotoxin, payload, or warhead) to an antibody whose antigen is a tumor associated antigen—i.e., an antigen expressed by a cancer cell. The antibody, upon binding to the antigen, delivers the ADC to the cancer site. There, cleavage of the linker or degradation of the antibody releases the drug, which typically is a cytotoxic agent capable of killing the cancer cell. Frequently, the ADC is internalized by endocytosis into the target cell and release of the drug takes place inside it. While the ADC is circulating in the blood, the drug is held inactive because of its linkage to the antibody. For a review on ADCs, see Schrama et al., Nature Rev. Drug Disc. 2006, 5, 147-159.

Targeted therapy by multiple non-redundant molecular pathways can enhance anticancer immunotherapy. However, not all combinations have acceptable profiles. There remains a need for combination therapies with an acceptable safety profile and high efficacy that enhance anticancer immune responses compared to monotherapy and other immunotherapy combinations.

U.S. Provisional Application Ser. No. 62/344866, filed Jun. 6, 2016, discloses a combination of an anti-PD-1 antibody and an anti-CD30 antibody or a conjugate of an anti-CD30 antibody for the treatment of lymphoma. The conjugate can be brentuximab vedotin (ADCETRIS®), in which the attached drug is monomethyl auristatin E (MMAE).

### BRIEF SUMMARY OF THE INVENTION

The present disclosure relates to a method of treating a subject afflicted with a tumor, comprising administering to the subject: (a) an antibody or an antigen-binding portion thereof that binds specifically to a Programmed Death-1 (PD-1) receptor and inhibits PD-1 activity (“anti-PD-1 antibody”) and (b) antibody-drug conjugate of an anti-mesothelian antibody (“anti-mesothelin ADC”). In some embodiments, the cancer is non-small cell lung cancer (NSCLC), ovarian cancer, mesothelioma, pancreatic cancer, or gastric cancer.

In some embodiments, the cancer comprises one or more cells that express mesothelin. In certain embodiments, at least 1% of the cancer cells express mesothelin.

In some embodiments, the anti-PD-1 antibody cross-competes with nivolumab for binding to human PD-1. In some embodiments, the anti-PD-1 antibody binds to the same epitope as nivolumab. In one particular embodiment, the anti-PD-1 antibody is nivolumab.

In certain embodiments, the anti-PD-1 antibody is administered at a dose of at least about 3 mg/kg body weight once about every 2 weeks. In some embodiments, the anti-mesothelin ADC is administered at a dose of 1.8 mg/kg body weight once about every 3 weeks.

In some embodiments, the cancer comprises one or more cells that express PD-L1, PD-L2, or both. Preferably, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the tumor cells express PD-L1 or PD-L2, or both.

In some embodiments, the subject received at least one prior chemotherapy treatment. In certain embodiments, the subject was not responsive to a prior chemotherapy treatment.

In some embodiments, the method further comprises administering a stem cell transplant to the patient after administering the anti-PD-1 antibody and the anti-mesothelin antibody.

The present disclosure is further directed to a kit for treating a subject afflicted with a cancer, the kit comprising: (a) a dosage ranging from about 4 mg to about 500 mg of an anti-PD-1 antibody; (b) a dosage ranging from about 0.1 mg to about 500 mg of an anti-mesothelin ADC; and (c) instructions for using the anti-PD-1 antibody and the anti-mesothelin ADC in the method.

The present disclosure is further directed to a kit for treating a subject afflicted with a cancer, the kit comprising: (a) a dosage ranging from about 4 mg to about 500 mg of an anti-PD-1 antibody; (b) a dosage ranging from about 0.1 mg to about 500 mg of an anti-mesothelin ADC having a structure according to formula (I) hereinbelow; and (c) instructions for using the anti-PD-1 antibody and the anti-mesothelin ADC in the method.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the tumor cells express mesothelin.

### BRIEF DESCRIPTION OF THE DRAWING(S)

FIG. 1 shows a schematic drawing of the PD-1 pathway and the mode of action of nivolumab.

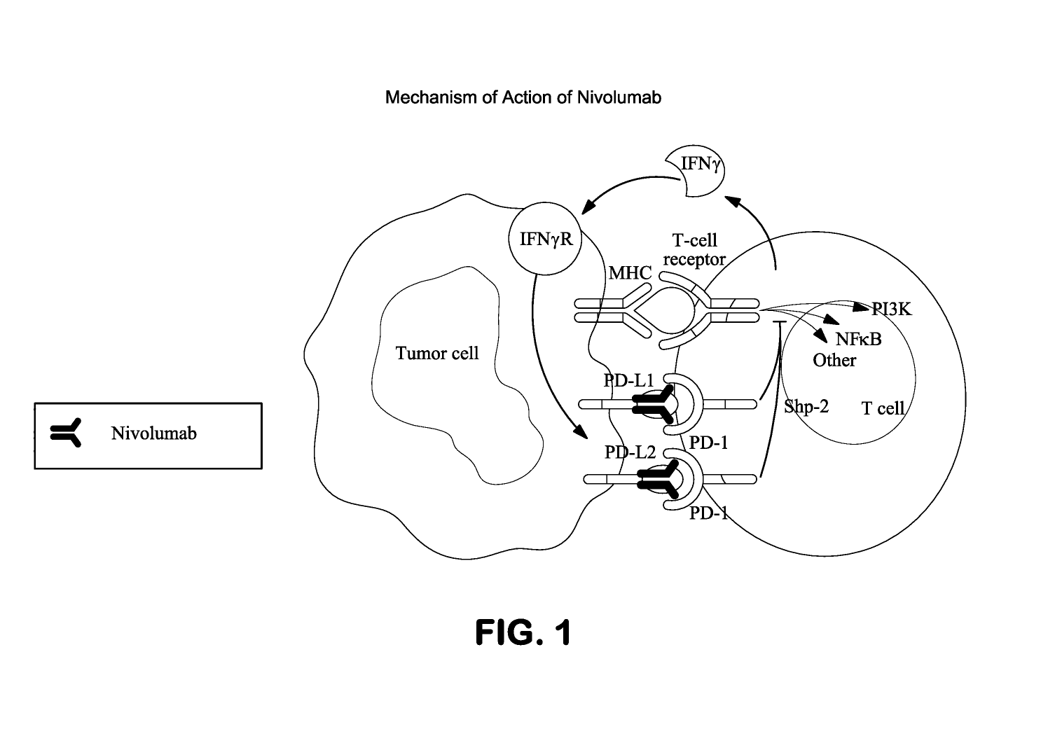

### DETAILED DESCRIPTION OF THE INVENTION

This invention relates to methods for treating a cancer in a subject comprising administering to the subject an anti-Programmed Death-1 (PD-1) antibody and an anti-mesothelin ADC.

### DEFINITIONS

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

“Administering” refers to the physical introduction of a therapeutic agent to a subject, using any of the various methods and delivery systems known to those skilled in the art. Exemplary routes of administration for the anti-PD-1 antibody include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 can, however, have cross-reactivity to other antigens, such as PD-1 molecules from different species. Moreover, an isolated antibody can be substantially free of other cellular material and/or chemicals. In one embodiment, an antibody includes a conjugate attached to another agent (e.g., small molecule drug), such as an anti-mesothelin ADC.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs can be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-PD-1 antibody binds specifically to PD-1 and an anti-mesothelin antibody binds specifically to mesothelin.

An “antigen-binding portion” of an antibody (also called an “antigen-binding fragment”) refers to one or more fragments of an antibody that retain the ability to bind specifically to the antigen bound by the whole antibody.

A “cancer” refers a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a non-Hodgkin's Lymphoma refers to a tumor that is the result of a metastasized non-Hodgkin's Lymphoma. Because the distal tumor is derived from the pre-metastasis tumor, the “derived from” tumor can also comprise the pre-metastasis tumor, e.g., a tumor derived from a non-Hodgkin's Lymphoma can comprise a non-Hodgkin's Lymphoma.

The term “immunotherapy” refers to the treatment of a subject afflicted with, or at risk of contracting or suffering a recurrence of, a disease by a method comprising inducing, enhancing, suppressing or otherwise modifying an immune response.

“Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1 (PD-L1)” is one of two cell surface glycoprotein ligands for PD-1 (the other being PD-L2) that down regulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

A “subject” includes any human or nonhuman animal. The term “nonhuman animal” includes, but is not limited to, vertebrates such as nonhuman primates, sheep, dogs, and rodents such as mice, rats and guinea pigs. In some embodiments, the subject is a human. The terms, “subject” and “patient” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “subtherapeutic dose” means a dose of a therapeutic compound (e.g., an antibody) that is lower than the usual or typical dose of the therapeutic compound when administered alone for the treatment of a hyperproliferative disease (e.g., cancer).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent inhibits cell growth or tumor growth by at least about 20%, by at least about 30%, by at least about 40%, by at least about 50%, by at least about 60%, by at least about 70%, or by at least about 80% relative to untreated subjects.

In other embodiments of the invention, tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for “immune-related” response patterns.

An “immune-related” response pattern refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

A therapeutically effective amount of a drug includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

The term “weight based dose” as referred to herein means that a dose that is administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration.

The use of the term “fixed dose” with regard to a method of the invention means that two or more different antibodies in a single composition (e.g., anti-PD-1 antibody and anti-mesothelin antibody) are present in the composition in particular (fixed) ratios with each other. In some embodiments, the fixed dose is based on the weight (e.g., mg) of the antibodies. In certain embodiments, the fixed dose is based on the concentration (e.g., mg/ml) of the antibodies. In some embodiments, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first antibody (e.g., anti-PD-1 antibody) to mg second antibody (e.g., anti-mesothelin antibody). For example, the 3:1 ratio of an anti-PD-1 antibody and an anti-mesothelin antibody can mean that a vial can contain about 240 mg of the anti-PD-1 antibody and 80 mg of the anti-mesothelin antibody or about 3 mg/ml of the anti-PD-1 antibody and 1 mg/ml of the anti-mesothelin antibody.

The use of the term “flat dose” with regard to the methods and dosages of the invention means a dose that is administered to a patient without regard for the weight or body surface area (BSA) of the patient. The flat dose is therefore not provided as a mg/kg dose, but rather as an absolute amount of the agent (e.g., the anti-mesothelin antibody and/or anti-PD-1 antibody). For example, a 60 kg person and a 100 kg person would receive the same dose of an antibody (e.g., 240 mg of an anti-PD-1 antibody).

The use of the alternative (e.g.,“or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days ±one day, i.e., every six days to every eight days. “Once about every two weeks” can include every fourteen days ±three days, i.e., every eleven days to every seventeen days. Similar approximations apply, for example, to once about every three weeks, once about every four weeks, once about every five weeks, once about every six weeks and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

### METHODS OF THE INVENTION

The present invention is directed to a method for treating a tumor or a subject afflicted with a tumor comprising administering to the subject a therapeutically effective amount of an antibody or an antigen-binding portion thereof that binds specifically to a Programmed Death-1 (PD-1) receptor and inhibits PD-1 activity (“anti-PD-1 antibody”) or an antibody or an antigen-binding portion thereof that binds specifically to a Programmed Death Ligandl1 (PD-L1) receptor and inhibits PD-L1 activity (“anti-PD-L1 antibody”) and a therapeutically effective amount of anti-mesothelin ADC. In some embodiments, the cancer is non-small cell lung cancer (NSCLC), ovarian cancer, mesothelioma, pancreatic cancer, or gastric cancer.

In certain embodiments, the subject has received one, two, three, four, five or more prior cancer treatments. In other embodiments, the subject is treatment-naive. In some embodiments, the subject has progressed on other cancer treatments. In some embodiments, the tumor has reoccurred. In some embodiments, the tumor is metastatic. In other embodiments, the tumor is not metastatic.

In other embodiments, the present methods comprise administering an effective amount of an anti-PD-1 antibody and an effective amount of an anti-mesothelin ADC. An effective amount of an anti-PD-1 antibody and/or an anti-mesothelin ADC can be a flat dose or a weight based dose.

In embodiments, the invention includes a method of treating a cancer or a subject afflicted with cancer comprising administering an anti-PD-1 antagonist in combination with an anti-mesothelin ADC. An “anti-PD-1 antagonist” as referred herein includes any molecule that inhibits interaction between PD-1 (receptor) and PD-L1 (ligand) such that the signal pathway of PD-1/PD-L1 is blocked. In other embodiments, an anti-PD-1 antagonist is a PD-1-Fc fusion protein. In certain embodiments, an anti-PD-1 antagonist includes an anti-PD-1 fusion protein, an antisense molecule, a small molecule, a ribozyme, or a nanobody that inhibits or prevents interaction between PD-1 and PD-L1.

In certain embodiments, the therapy of the present invention (e.g., administration of an anti-PD-1 antibody and the anti-mesothelin ADC) effectively increases the duration of survival of the subject. For example, the duration of survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months or at least about 1 year or more when compared to another subject treated with only either another therapy or, only one of the two members of the combination therapy alone (e.g., an anti-PD-1 antibody alone) or an alternative combination therapy. In still other embodiments, the combination therapy of an anti-PD-1 antibody (e.g., Nivolumab or Pembrolizumab) and an anti-mesothelin ADC increases the duration of survival of the subject at a level higher than (about one month higher than, about two months higher than, about three months higher than, about four months higher than, about five months higher than, about six months higher than, about seven months higher than, about eight months higher than, about nine months higher than, about ten months higher than, about eleven months higher than, or about one year higher than the duration of survival of the subject using a combination therapy of an anti-PD-L1 antibody and a different agent.

In certain embodiments, the therapy of the present invention effectively increases the duration of progression-free survival of the subject. For example, the progression free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months or at least about 1 year when compared to another subject treated with only either another therapy or only one of the two members of the combination therapy alone (e.g., an anti-PD-1 antibody alone) or an alternative combination therapy.

In certain embodiments, the therapy of the present invention effectively increases the response rate in a group of subjects. For example, the response rate in a group of subjects is increased by at least about 2%, at least about 3%, at least about 4%, at least about 5%, at least about 10%, at least about 15%, at least about 20%, at least about 25%, at least about 30%, at last about 35%, at least about 40%, at least about 45%, at least about 50%, at least about 55%, at least about 60%, at least about 70%, at least about 75%, at least about 80%, at least about 85%, at least about 90%, at least about 95%, at least about 99% or at least about 100% when compared to another group of subjects treated with only either another therapy or, only one of the two members of the combination therapy alone (e.g., an anti-PD-1 antibody alone) or an alternative combination therapy.

In some embodiments, the anti-PD-1 and anti-mesothelin ADC are formulated for intravenous administration. In certain embodiments, the anti-PD-1 and anti-mesothelin ADC are administered sequentially. In embodiments, the anti-PD-1 and anti-mesothelin ADC are administered within 30 minutes of each other. In one embodiment, the anti-PD-1 antibody or antigen-binding portion thereof is administered before the anti-mesothelin ADC. In another embodiment, the anti mesothelin ADC is administered before the anti-PD-1 antibody or antigen-binding portion thereof. In another embodiment, the anti-PD-1 antibody or antigen-binding portion thereof and the anti-mesothelin ADC are administered concurrently in separate compositions. In a further embodiment, the anti-PD-1 antibody or antigen-binding portion thereof and the anti-mesothelin ADCare admixed as a single composition for concurrent administration.

In some embodiments, the anti-PD-1 antibody and anti-mesothelin ADC are administered in a fixed dose.

### Anti-PD-1 and Anti-PD-L1 Antibodies

The combination therapy of the present invention can utilize an anti-PD-1 antibody or an antigen-binding fragment thereof. PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down regulate T cell activation and cytokine secretion upon binding to PD-1. Inhibition of the PD-1/PD-L1 interaction mediates potent antitumor activity in preclinical models.

HuMAbs that bind specifically to PD-1 with high affinity have been disclosed in U.S. Pat. No. 8,008,449. Other anti-PD-1 mABs have been described in, for example, U.S. Pat. Nos. 6,808,710, 7,488,802, 8,168,757 and 8,354,509, and PCT Publication No. WO 2012/145493. Each of the anti-PD-1 HuMAbs disclosed in U.S. Pat. No. 8,008,449 has been demonstrated to exhibit one or more of the following characteristics: (a) binds to human PD-1 with a KD of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-y production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates antibody responses; and/or (j) inhibits tumor cell growth in vivo. Anti-PD-1 antibodies usable in the present invention include mAbs that bind specifically to human PD-1 and exhibit at least one, at least two, at least three, at least four or at least five of the preceding characteristics.

In one embodiment, the anti-PD-1 antibody is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). Nivolumab has shown activity in a variety of advanced solid tumors including renal cell carcinoma (renal adenocarcinoma, or hypernephroma), melanoma, and non-small cell lung cancer (NSCLC) (Topalian et al., 2012a; Topalian et al., 2014; Drake et al., 2013; WO 2013/173223). In another embodiment, the anti-PD-1 antibody or fragment thereof cross-competes with nivolumab. In some embodiments, the anti-PD-1 antibody binds to the same epitope as nivolumab. In certain embodiments, the anti-PD-1 antibody has the same CDR regions as nivolumab.

Nivolumab monotherapy has been extensively studied in advanced melanoma, NSCLC, renal cell carcinoma, and classical Hodgkin lymphoma patients with body weight-based dosing (mg/kg) and is currently approved at a dose of 3 mg/kg every 2 weeks (Q2W) in these populations.

In another embodiment, the anti-PD-1 antibody or fragment thereof cross-competes with pembrolizumab. In some embodiments, the anti-PD-1 antibody binds to the same epitope as pembrolizumab. In certain embodiments, the anti-PD-1 antibody has the same CDR regions as pembrolizumab. In another embodiment, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. No. 8,900,587; see also http://www.cancer.gov/drugdictionary?cdrid=695789 (last accessed: Dec. 14, 2014). Pembrolizumab has been approved by the FDA for the treatment of relapsed or refractory melanoma and advanced NSCLC.

In other embodiments, the anti-PD-1 antibody or fragment thereof cross-competes with MEDI0608. In some embodiments, the anti-PD-1 antibody binds to the same epitope as MEDI0608. In certain embodiments, the anti-PD-1 antibody has the same CDR regions as MEDI0608. In other embodiments, the anti-PD-1 antibody is MEDI0608 (formerly AMP-514), which is a monoclonal antibody against the PD-1 receptor. MEDI0608 is described, for example, in U.S. Pat. No. 8,609,089,B2 or in http://www.cancer.gov/drugdictionary?cdrid=756047 (last accessed Dec. 14, 2014).

In certain embodiments, an immune checkpoint inhibitor is AMP-224, which is a B7-DC Fc fusion protein. AMP-224 is discussed in U.S. Publ. No. 2013/0017199 or in http://www.cancer.gov/publications/dictionaries/cancer-drug?cdrid=700595 (last accessed Jul. 8, 2015).

In other embodiments, the anti-PD-1 antibody or fragment thereof cross-competes with BGB-A317. In some embodiments, the anti-PD-1 antibody binds to the same epitope as BGB-A317. In certain embodiments, the anti-PD-1 antibody has the same CDR regions as BGB-A317. In certain embodiments, the anti-PD-1 antibody is BGB-A317, which is a humanized monoclonal antibody. BGB-A317 is described in U.S. Publ. No. 2015/0079109.

Anti-PD-1 antibodies usable in the disclosed methods also include isolated antibodies that bind specifically to human PD-1 and cross-compete for binding to human PD-1 with nivolumab (see, e.g., U.S. Pat. No. 8,008,449; WO 2013/173223). The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of nivolumab by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1 with, or bind to the same epitope region of human PD-1 as nivolumab are mAbs. For administration to human subjects, these cross-competing antibodies can be chimeric antibodies, or can be humanized or human antibodies. Such chimeric, humanized or human mAbs can be prepared and isolated by methods well known in the art.

Anti-PD-1 antibodies usable in the methods of the disclosed invention also include antigen-binding portions of the above antibodies. It has been amply demonstrated that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)2 fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an antibody.

In certain embodiments, the anti-PD-1 antibody or antigen-binding portion thereof comprises a heavy chain constant region which is of a human IgG1 or IgG4 isotype. In certain other embodiments, the sequence of the IgG4 heavy chain constant region of the anti-PD-1 antibody or antigen-binding portion thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In yet other embodiments, the antibody comprises a light chain constant region which is a human kappa or lambda constant region. In other embodiments, the anti-PD-1 antibody or antigen-binding portion thereof is a mAb or an antigen-binding portion thereof. In certain embodiments of any of the therapeutic methods described herein comprising administration of an anti-PD-1 Ab, the anti-PD-1 antibody is nivolumab. In other embodiments, the anti-PD-1 antibody is pembrolizumab. In other embodiments, the anti-PD-1 antibody is chosen from the human antibodies 17D8, 2D3, 4H1, 4A11, 7D3 and 5F4 described in U.S. Pat. No. 8,008,449. In still other embodiments, the anti-PD-1 antibody is MEDI0608 (formerly AMP-514), AMP-224, or BGB-A317.

In other embodiments, the anti-PD-1 antibody or antigen-binding portion thereof is a chimeric, humanized or human monoclonal antibody or a portion thereof. In certain embodiments for treating a human subject, the antibody is a humanized antibody. In other embodiments for treating a human subject, the antibody is a human antibody. Antibodies of an IgG1, IgG2, IgG3 or IgG4 isotype can be used.

In certain embodiments, an anti-PD-1 antibody used in the methods can be replaced with another PD-1 or anti-PD-L1 antagonist. For example, because an anti-PD-L1 antibody prevents interaction between PD-1 and PD-L1, thereby exerting similar effects to the signaling pathway of PD-1, an anti-PD-L1 antibody can replace the use of an anti-PD-1 antibody in the methods disclosed herein. Therefore, in one embodiment, the present invention is directed to a method for treating a subject afflicted with a tumor comprising administering to the subject a therapeutically effective amount an anti-PD-L1 antibody and an anti-mesothelin ADC.

In certain embodiments, the anti-PD-L1 antibody is BMS-936559 (formerly 12A4 or MDX-1105) (see, e.g., U.S. Pat. No. 7,943,743; WO 2013/173223).

In other embodiments, the anti-PD-L1 antibody is MPDL3280A (also known as RG7446) (see, e.g., Herbst et al. (2013) J Clin Oncol 31(suppl):3000. Abstract.; U.S. Pat. No. 8,217,149).

In other embodiments, the anti-PD-L1 antibody is MEDI4736 (also called Durvalumab; Khleif (2013) In: Proceedings from the European Cancer Congress 2013; Sep. 27-Oct. 1, 2013; Amsterdam, The Netherlands. Abstract 802, See U.S. Pat. No. 8,779,108 or US 2014/0356353, filed May 6, 2014).

In further embodiments, the anti-PD-L1 antibody is MSB0010718C (also called Avelumab; See US 2014/0341917).

In certain embodiments, the anti-PD-L1 antibodies cross-compete for binding to human PD-L1 with, or bind to the same epitope region of human PD-L1 as the above-references PD-L1 antibodies. In other embodiments, the anti-PD-L1 antibodies useful for the combination therapy with an anti-mesothelin ADC are mAbs. For administration to human subjects, these cross-competing antibodies can be chimeric antibodies, or can be humanized or human antibodies. Such chimeric, humanized or human mAbs can be prepared and isolated by methods well known in the art.

### Anti-Mesothelin Antibodies and their ADCS

Mesothelin is a glycosylphosphatidylinositol (GPI)-anchored cell surface protein which is highly expressed in a number of malignancies, but its expression is relatively restricted in normal tissue. The antigen is normally expressed at the cell surface of mesothelial cells of the pleura, pericardium, and peritoneum. Mesothelin is a potential target for antibody-based cancer therapy due to its high expression in multiple tumors, including mesothelioma, ovarian cancer, pancreatic cancer, non-small cell lung cancer (NSCLC), triple negative breast cancer, gastric carcinoma, and other cancers, which are associated with the prevalence of mesothelin expression, and correlation of efficacy with mesothelin expression has been observed in pre-clinical models.

The precise function of mesothelin remains unknown. Mesothelin knockout mice have no obvious phenotype and produce normal offspring without anatomical abnormalities, suggesting that it is a nonessential protein. However, it has been suggested that mesothelin plays a role in adhesion and metastasis because of its ability to bind to the cancer antigen, CA125 (MUC-16). The data support that CA125 plays a role in mediating heterotypic cell adhesion whereby mesothelin expressed on the peritoneal lining would bind to CA125-positive ovarian tumor cells leading to their metastasis. Research continues to understand the role of mesothelin in cell adherence, cell survival/proliferation, tumor progression, and chemoresistance.

In some embodiments, the anti-mesothelin antibody in the ADC is antibody 6A4 or an antibody having the same heavy and light chain CDR1, CDR2, and CDR3 sequences as antibody 6A4. Antibody 6A4 is disclosed in Terrett et al., U.S. Pat. No. 8,268,970 B2 (2012), the disclosure of which is incorporated herein by reference.

Other anti-mesothelin antibodies that can be used in ADCs of this invention are disclosed in Terrett et al., U.S. Pat. No. 8,268,970 B2 (2012); Ho et al., US 2015/0274836 A1 (2015); Matsura et al., WO 2011/074621 A1 (2011); Fanslow, III et al., US 2014/0004121 A1 (2014); Pastan et al., US 2009/0047211 A1 (2009); Ho et al., U.S. Pat. No. 9,409.992 B2 (2016); U.S. Pat. No. 9,084,829 B2 (2015); Kahnert et al., U.S. Pat. No. 9023,351 B2 (2015); Dennis et al., U.S. Pat. No. 8,911,732 B2 (2014); Ho et al., U.S. Pat. No. 8,460,660 (B2); Dimitrov et al., U.S. Pat. No. 8,357,783 (B2); Ebel et al., U.S. Pat. No. 7,952,426 (2009); Pastan et al., U.S. Pat. No. 7,081,518 B1 (2006); and Pastan et al., U.S. Pat. No. 6,809,184 B1 (2004).

Various therapeutic agents also can be used can be used as the drug in the ADC, such as:
(a) enediynes such as calicheamicin (see, e.g., Lee et al., J. Am. Chem. Soc. 1987, 109, 3464 and 3466) and uncialamycin (see, e.g., Davies et al., WO 2007/038868 A2 (2007); Chowdari et al., U.S. Pat. No. 8,709,431 B2 (2012); and Nicolaou et al., WO 2015/023879 A1 (2015));
(b) tubulysins (see, e.g., Domling et al., U.S. Pat. No. 7,778,814 B2 (2010); Cheng et al., U.S. Pat. No. 8,394,922 B2 (2013); and Cong et al., U.S. Pat. No. 8,980,824 B2 (2015));
(c) DNA alkylators such as analogs of CC-1065 and duocarmycin (see, e.g., Boger, U.S. Pat. No. 6,5458,530 B1 (2003); Sufi et al., U.S. Pat. No. 8,461,117 B2 (2013); and Zhang et al., U.S. Pat. No. 8,852,599 B2 (2014));
(d) epothilones (see, e.g., Vite et al., US 2007/0275904 A1 (2007) and US RE42930 E (2011));
(e) auristatins (see, e.g., Senter et al., U.S. Pat. No. 6,844,869 B2 (2005) and Doronina et al., U.S. Pat. No. 7,498,298 B2 (2009); especially monomethyl auristatin E or MMAE);
(f) pyrrolobezodiazepine (PBD) dimers (see, e.g., Howard et al., US 2013/0059800 A1(2013); US 2013/0028919 A1 (2013); and WO 2013/041606 A1 (2013));
(g) maytansinoids such as DM1 and DM4 (see, e.g., Chari et al., U.S. Pat. No. 5,208,020 (1993) and Amphlett et al., U.S. Pat. No. 7,374,762 B2 (2008)); and
(h) tetrahydroisoquinoline (THIQ) dimers (see, e.g., Zhang et al., US 2016/0200742 A1 (2016) and McDonald et al. US 2016/0199510 A1 (2016)).

Preferably, the therapeutic agent is a tubulysin analog, as disclosed in Cheng et al., U.S. Pat. No. 8,394,922 B2 (2013), the disclosure of which is incorporated herein by reference. A preferred tubulysin analog has a structure represented by formula (A):

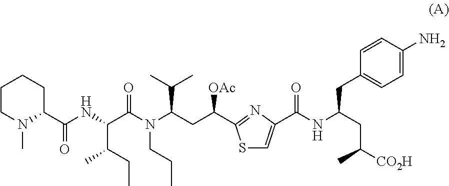 [SMILES: CCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)C[C@H](C)C(=O)O)cs1)C(C)C]

In a preferred embodiment, the anti-mesothelin ADC has a structure represented by formula (I), hereinafter also referred to as “ADC (I)”.

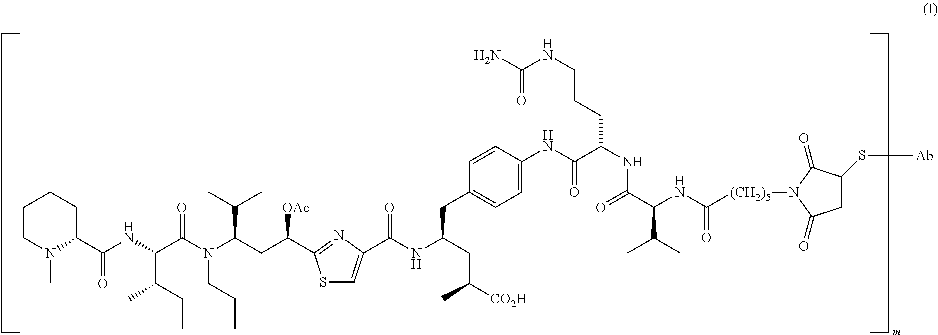

wherein
m is 1, 2, 3, or 4 and
Ab is an anti-mesothelin antibody, preferably antibody 6A4.

The preparation of ADCs according to formula (I), in particular ADC (I), is disclosed in Cheng et al., U.S. Pat. No. 8,394,922 B2 (2013).

The mesothelin-directed antibody (especially antibody 6A4) portion of ADC (I) can be a fully human immunoglobulin G1 (IgG1) monoclonal antibody that specifically binds mesothelin and exhibits high affinity binding to mesothelin-expressing human tumor cells. The monoclonal antibody targets mesothelin, a 40 kDa GPI anchored cell surface protein, which is highly expressed in a number of cancers. The monoclonal anti-mesothelin antibody is conjugated to tubulysin analog (A), which is a synthetic small molecule cytotoxic via a valine-citrulline linker. The tubulysins are a family of complex tetra-peptides with promising potent sub-nanomolar cytotoxic activity against multi-drug resistant tumors. Mechanism of action studies have shown that tubulysins disrupt microtubule assembly. Since microtubules are involved in separation of the mitotic figures during metaphase, tubulin binding agents such as tubulysins inhibit cell division, resulting in apoptosis. Upon binding to cell surface mesothelin, the ADC is internalized and traffics to lysosomes where the linker is cleaved by an enzyme such as cathepsin B, releasing the tubulysin analog. The analog in turn binds tubulin resulting in inhibition of cellular proliferation and tumor cell death.

Other anti-mesothelin ADCs that can be used in this invention include anetumab ravtansine (also known as BAY 94-9343; Golfier et al., Mol. Cancer Ther. 2014, 13 (6), 1537) and DMOT4039A (Weekes et al., Mol. Cancer Ther. 2016, 15 (3), 439).

### Cancer and Standard-of-Care Therapies

In some embodiments, the methods disclosed herein are used in place of standard of care therapies. In certain embodiments, a standard of care therapy is used in combination with any method disclosed herein. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES®, 2014).

### Pharmaceutical Compositions and Dosages

Therapeutic agents of the present invention can be constituted in a composition, e.g., a pharmaceutical composition containing an antibody and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. In some embodiments, the carrier for a composition containing an antibody is suitable for intravenous, intramuscular, subcutaneous, parenteral, spinal or epidermal administration (e.g., by injection or infusion). A pharmaceutical composition of the invention can include one or more pharmaceutically acceptable salts, anti-oxidant, aqueous and non-aqueous carriers, and/or adjuvants such as preservatives, wetting agents, emulsifying agents and dispersing agents.

Dosage regimens are adjusted to provide the optimum desired response, e.g., a maximal therapeutic response and/or minimal adverse effects. In some embodiments, the anti-PD-1 antibody is administered at a weight-based dose. For administration of an anti-PD-1 antibody, the dosage can range from at least about 0.01 to at least about 20 mg/kg, from at least about 0.1 to at least about 10 mg/kg, of the subject's body weight. For example, dosages can be at least about 0.1, at least about 0.3, at least about 1, at least about 2, at least about 3, at least about 5 or at least about 10 mg/kg body weight, and at least about 0.3, at least about 1, at least about 2, at least about 3, or at least about 5 mg/kg body weight. In certain embodiments, the dosage of the anti-PD-1 antibody is 3 mg/kg body weight. In certain embodiments, an anti-PD-1 antibody is administered at a flat dose. In embodiments, the flat dose of the anti-PD-1 antibody is a dose (e.g., flat dose) of at least about 100-300 mg, such as, at least about 200-300 mg, at least about 220-260 mg, at least about 230-250 mg or at least about 240 mg, such as at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg or at least about 300 mg. In one embodiment, the anti-PD-1 antibody is a dose (e.g., flat dose) of at least about 240 mg.

In some embodiments, the anti-PD-1 antibody is administered in a fixed dose with the anti-mesothelin ADC. In some embodiments, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg anti-PD-1 antibody to mg anti-mesothelin ADC.

The dosing schedule is typically designed to achieve exposures that result in sustained receptor occupancy (RO) based on typical pharmacokinetic properties of an antibody. An exemplary treatment regime entails administration once per week, once about every 2 weeks, once about every 3 weeks, once about every 4 weeks, once about a month, once about every 3-6 months or longer. In certain embodiments, an anti-PD-1 antibody such as nivolumab is administered to the subject once about every 2 weeks. In other embodiments, the antibody is administered once about every 3 weeks. The dosage and scheduling can change during a course of treatment.

When used in combinations with other anti-cancer agents, the dosage of an anti-PD-1 antibody can be lowered compared to the monotherapy dose. Dosages of nivolumab that are lower than the typical 3 mg/kg, but not less than 0.001 mg/kg, are subtherapeutic dosages. The subtherapeutic doses of an anti-PD-1 antibody used in the methods herein are higher than 0.001 mg/kg and lower than 3mg/kg. In some embodiments, a subtherapeutic dose is about 0.001 mg/kg-about 1 mg/kg, about 0.01 mg/kg-about 1 mg/kg, about 0.1 mg/kg-about 1 mg/kg, or about 0.001 mg/kg-about 0.1 mg/kg body weight. In some embodiments, the subtherapeutic dose is at least about 0.001 mg/kg, at least about 0.005 mg/kg, at least about 0.01 mg/kg, at least about 0.05 mg/kg, at least about 0.1 mg/kg, at least about 0.5 mg/kg, or at least about 1.0 mg/kg body weight. Receptor-occupancy data from 15 subjects who received 0.3 mg/kg to 10 mg/kg dosing with nivolumab indicate that PD-1 occupancy appears to be dose-independent in this dose range. Across all doses, the mean occupancy rate was 85% (range, 70% to 97%), with a mean plateau occupancy of 72% (range, 59% to 81%). In some embodiments, 0.3 mg/kg dosing can allow for sufficient exposure to lead to maximal biologic activity.

In some embodiments, the anti-mesothelin ADC is administered at a weight-based dose. The dosage can range from about 0.01 to about 20 mg/kg, about 0.05 to about 20 mg/kg, about 0.1 to about 20 mg/kg, about 0.1 to about 15 mg/kg, about 0.1 to about 10 mg/kg, about 0.1 to about 5 mg/kg, about 0.1 to about 4 mg/kg, about 0.1 to about 3 mg/kg, about 0.1 to about 2 mg/kg, about 1 to about 10 mg/kg, about 1 to about 10 mg/kg, about 1 to about 8 mg/kg, about 1 to about 5 mg/kg, about 1 to about 3 mg/kg, about 1 to about 2 mg/kg of the subject's body weight. For example, dosages can be about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg or about 20 mg/kg of the subject's body weight.

In some embodiments, the dosage of the anti-mesothelin ADC is 0.1 mg/kg body weight. In other embodiments, the dosage of the anti-mesothelin ADC is 0.2 mg/kg body weight. In other embodiments, the dosage of the anti-mesothelin ADC is 0.3 mg/kg body weight. In other embodiments, the dosage of the anti-mesothelin ADC is 0.4 mg/kg body weight. In other embodiments, the dosage of the anti-mesothelin ADC is 0.5 mg/kg body weight. In other embodiments, the dosage of the anti-mesothelin ADC is 0.6 mg/kg body weight. In other embodiments, the dosage of the anti-mesothelin ADC is 0.7 mg/kg body weight. In other embodiments, the dosage of the anti-mesothelin ADC is 0.8 mg/kg body weight. In other embodiments, the dosage of the anti-mesothelin ADC is 0.9 mg/kg body weight. In other embodiments, the dosage of the anti-mesothelin ADC is 1.0 mg/kg body weight. In other embodiments, the dosage of the anti-mesothelin ADC is 1.1 mg/kg body weight. In other embodiments, the dosage of the anti-mesothelin ADC is 1.2 mg/kg body weight. In other embodiments, the dosage of the anti-mesothelin ADC is 1.3 mg/kg body weight. In other embodiments, the dosage of the anti-mesothelin ADC is 1.4 mg/kg body weight. In other embodiments, the dosage of the anti-mesothelin ADC is 1.5 mg/kg body weight. In other embodiments, the dosage of the anti-mesothelin ADC is 1.6 mg/kg body weight. In other embodiments, the dosage of the anti-mesothelin ADC is 1.7 mg/kg body weight. In other embodiments, the dosage of the anti-mesothelin ADC is 1.8 mg/kg body weight. In other embodiments, the dosage of the anti-mesothelin ADC is 1.9 mg/kg body weight. In other embodiments, the dosage of the anti-mesothelin ADC is 2.0 mg/kg body weight. In other embodiments, the dosage of the anti-mesothelin ADC is 2.1 mg/kg body weight. In other embodiments, the dosage of the anti-mesothelin ADC is 2.2 mg/kg body weight. In other embodiments, the dosage of the anti-mesothelin ADC is 2.3 mg/kg body weight. In other embodiments, the dosage of the anti-mesothelin ADC is 2.4 mg/kg body weight. In other embodiments, the dosage of the anti-mesothelin ADC is 2.5 mg/kg body weight. In other embodiments, the dosage of the anti-mesothelin ADC is about 5 mg/kg body weight. In other embodiments, the dosage of the anti-mesothelin ADC is about 10 mg/kg body weight.

In certain embodiments, an anti-mesothelin ADC is administered at a flat dose. In some embodiments, the flat dose of the anti-mesothelin ADC is a dose (e.g., flat dose) of at least about 1-1500 mg, at least about 10-1000 mg, such as, at least about 50-800 mg, at least about 100-600 mg, at least about 100-400 mg or at least about 100-200 mg, such as at least about 1 mg, at least about 3 mg, at least about 5 mg, at least about 8 mg, at least about 10 mg, at least about 20 mg, at least about 30 mg, at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 600 mg, at least about 700 mg, at least about 800 mg, at least about 900 mg, at least about 1000 mg, at least about 1100 mg, at least about 1200 mg, at least about 1300 mg, at least about 1400 mg, or at least about 1500 mg.

An exemplary treatment regime entails administration once per week, once about every 2 weeks, once about every 3 weeks, once about every 4 weeks, once about a month, once about every 3-6 months or longer. In certain embodiments, the anti-mesothelin ADC is administered once about every 3 weeks.

In some embodiments, a subtherapeutic dose of an anti-mesothelin ADC is used in the methods herein. The subtherapeutic dosages of an anti-mesothelin ADC used in the methods herein are higher than 0.001 mg/kg and lower than 10 mg/kg. In some embodiments, the subtherapeutic dose is about 0.001 mg/kg-about 10 mg/kg, about 0.01 mg/kg-about 10 mg/kg, about 0.01 mg/kg-about 1 mg/kg, about 0.1 mg/kg-about 1 mg/kg, or about 0.001 mg/kg-about 0.1 mg/kg body weight. In some embodiments, the subtherapeutic dose is at least about 0.001 mg/kg, at least about 0.005 mg/kg, at least about 0.01 mg/kg, at least about 0.05 mg/kg, at least about 0.1 mg/kg, at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, at least about 1 mg/kg, at least about 1.1 mg/kg, at least about 1.2 mg/kg, at least about 1.3 mg/kg, at least about 1.4 mg/kg, at least about 1.5 mg/kg, at least about 1.6 mg/kg, or at least about 1.7 mg/kg body weight.

In certain embodiments, at least about 0.1 to about 5 mg/kg of the anti-mesothelin ADC and at least about 240 mg of the anti-PD-1 antibody are administered to the subject once about every three weeks. In certain embodiments, at least about 0.1 mg/kg of the anti-mesothelin ADC and at least about 240 mg of the anti-PD-1 antibody are administered to the subject once about every three weeks. In certain embodiments, at least about 0.2 mg/kg of the anti-mesothelin ADC and at least about 240 mg of the anti-PD-1 antibody are administered to the subject once about every three weeks. In certain embodiments, at least about 0.3 mg/kg of the anti-mesothelin ADC and at least about 240 mg of the anti-PD-1 antibody are administered to the subject once about every three weeks. In certain embodiments, at least about 0.4 mg/kg of the anti-mesothelin ADC and at least about 240 mg of the anti-PD-1 antibody are administered to the subject once about every three weeks. In certain embodiments, at least about 0.5 mg/kg of the anti-mesothelin ADC and at least about 240 mg of the anti-PD-1 antibody are administered to the subject once about every three weeks. In certain embodiments, at least about 0.6 mg/kg of the anti-mesothelin ADC and at least about 240 mg of the anti-PD-1 antibody are administered to the subject once about every three weeks. In certain embodiments, at least about 0.7 mg/kg of the anti-mesothelin ADC and at least about 240 mg of the anti-PD-1 antibody are administered to the subject once about every three weeks. In certain embodiments, at least about 0.8 mg/kg of the anti-mesothelin ADC and at least about 240 mg of the anti-PD-1 antibody are administered to the subject once about every three weeks. In certain embodiments, at least about 0.9 mg/kg of the anti-mesothelin ADC and at least about 240 mg of the anti-PD-1 antibody are administered to the subject once about every three weeks. In certain embodiments, at least about 1 mg/kg of the anti-mesothelin ADC and at least about 240 mg of the anti-PD-1 antibody are administered to the subject once about every three weeks. In certain embodiments, at least about 1.1 mg/kg of the anti-mesothelin ADC and at least about 240 mg of the anti-PD-1 antibody are administered to the subject once about every three weeks. In certain embodiments, at least about 1.2 mg/kg of the anti-mesothelin ADC and at least about 240 mg of the anti-PD-1 antibody are administered to the subject once about every three weeks. In certain embodiments, at least about 1.3 mg/kg of the anti-mesothelin ADC and at least about 240 mg of the anti-PD-1 antibody are administered to the subject once about every three weeks. In certain embodiments, at least about 1.4 mg/kg of the anti-mesothelin ADC and at least about 240 mg of the anti-PD-1 antibody are administered to the subject once about every three weeks. In certain embodiments, at least about 1.5 mg/kg of the anti-meosothelin ADC and at least about 240 mg of the anti-PD-1 antibody are administered to the subject once about every three weeks. In certain embodiments, at least about 1.6 mg/kg of the anti-mesothelin ADC and at least about 240 mg of the anti-PD-1 antibody are administered to the subject once about every three weeks. In certain embodiments, at least about 1.7 mg/kg of the anti-mesothelin ADC and at least about 240 mg of the anti-PD-1 antibody are administered to the subject once about every three weeks. In certain embodiments, at least about 1.8 mg/kg of the anti-mesothelin ADC and at least about 240 mg of the anti-PD-1 antibody are administered to the subject once about every three weeks. In certain embodiments, at least about 1.9 mg/kg of the anti-mesothelin ADC and at least about 240 mg of the anti-PD-1 antibody are administered to the subject once about every three weeks. In certain embodiments, at least about 2 mg/kg of the anti-mesothelin ADC and at least about 240 mg of the anti-PD-1 antibody are administered to the subject once about every three weeks. In certain embodiments, at least about 3 mg/kg of the anti-mesothelin ADC and at least about 240 mg of the anti-PD-1 antibody are administered to the subject once about every three weeks. In certain embodiments, at least about 4 mg/kg of the anti-mesothelin ADC and at least about 240 mg of the anti-PD-1 antibody are administered to the subject once about every three weeks. In certain embodiments, at least about 5 mg/kg of the anti-mesothelin ADC and at least about 240 mg of the anti-PD-1 antibody are administered to the subject once about every three weeks. In certain embodiments, at least about 10 mg/kg of the anti-mesothelin ADC and at least about 240 mg of the anti-PD-1 antibody are administered to the subject once about every three weeks. In embodiments, the anti-ADC is according to formula (I). In some embodiments, the anti-PD-1 antibody is nivolumab.

In certain embodiments, the combination of an anti-PD-1 antibody (e.g., Nivolumab) and an anti-mesothelin ADC is administered intravenously to the subject once about every 3 weeks for a total of nine weeks. In some embodiments, the nine week cycle is repeated 3 or 4 times. In embodiments, the subject is treated with a combination of an anti-PD-1 antibody (e.g., Nivolumab) and an anti-mesothelin ADC every 3 weeks for a total of nine weeks and 3 nine-week cycles are performed. In embodiments, the subject is treated with a combination of an anti-PD-1 antibody (e.g., Nivolumab) and an anti-mesothelin ADC every 3 weeks for a total of nine weeks and 4 nine-week cycles are performed. In embodiments, a subject is treated with the anti-PD-1 antibody for 12 nine-week cycles.

Treatment is continued as long as clinical benefit is observed or until unacceptable toxicity or disease progression occurs. In certain embodiments, the anti-PD-1 antibody can be administered at the dosage that has been shown to produce the highest efficacy as monotherapy in clinical trials, e.g., about 3 mg/kg of nivolumab administered once about every three weeks (Topalian et al., 2012 N Engl J Med 366:2443-54; Topalian et al., 2012 Curr Opin Immunol 24:207-12), at a flat dose of 240 mg, or at a significantly lower dose, i.e., at a subtherapeutic dose.

In certain embodiments, the subject is treated with a combination of an anti-PD-1 antibody and an anti-mesothelin ADC once about every 3 weeks for a set period of time followed by a monotherapy of an anti-PD-1 antibody or a monotherapy of an anti-mesothelin ADC. In some embodiments, the subject is treated with a combination of an anti-PD-1 antibody and an anti-mesothelin ADC once about every 3 weeks for about 6 weeks followed by a monotherapy of an anti-PD-1 antibody or a monotherapy of an anti-mesothelin ADC. In some embodiments, the subject is treated with a combination of an anti-PD-1 antibody and an anti-mesothelin ADC once about every 3 weeks for about 9 weeks followed by a monotherapy of an anti-PD-1 antibody or a monotherapy of an anti-mesothelin ADC. In some embodiments, the subject is treated with a combination of an anti-PD-1 antibody and an anti-mesothelin ADC once about every 3 weeks for about 12 weeks followed by a monotherapy of an anti-PD-1 antibody or a monotherapy of an anti-mesothelin ADC. In some embodiments, the subject is treated with a combination of an anti-PD-1 antibody and an anti-mesothelin ADC once about every 3 weeks for about 24 weeks followed by a monotherapy of an anti-PD-1 antibody or a monotherapy of an anti-mesothelin ADC. In some embodiments, the subject is treated with a combination of an anti-PD-1 antibody and an anti-mesothelin ADC once about every 3 weeks for about 48 weeks followed by a monotherapy of an anti-PD-1 antibody or a monotherapy of an anti-mesothelin ADC. The monotherapy of the anti-PD-1 antibody can be administered by any route disclosed herein at any dose disclosed herein. In one embodiment, the monotherapy of the anti-PD-1 antibody is administered intravenously at a flat dose of 240 mg. In another embodiment, the monotherapy of the anti-PD-1 antibody is administered intravenously at a dose of 3 mg/kg or 6 mg/kg. The monotherapy of the anti-mesothelin ADC can be administered by any route disclosed herein at any dose disclosed herein. In one embodiment, the monotherapy of the anti-mesothelin ADC is administered intravenously at a dose of 1.8 mg/kg.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and until the patient shows partial or complete amelioration of symptoms of disease. Thereafter, the patient can be administered a prophylactic regime.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present invention can be administered via one or more routes of administration using one or more of a variety of methods well known in the art. As will be appreciated by the skilled artisan, the route and/or mode of administration will vary depending upon the desired results.

### Kits

Also within the scope of the present invention are kits comprising an anti-PD-1 antibody and an anti-mesothelin ADC for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a cancer, the kit comprising: (a) a dosage ranging from about 4 mg to about 500 mg of an anti-PD-1 antibody or antigen-binding portion thereof; and (b) a dosage ranging from about 0.1 mg to about 500 mg of an anti-mesothelin ADC and (c) instructions for using the anti-PD-1 antibody and the anti-mesothelin ADC in any of the combination therapy methods disclosed herein. In certain embodiments, the anti-PD-1 Ab, the anti-mesothelin ADC can be co-packaged in unit dosage form. In certain embodiments for treating human patients, the kit comprises an anti-human PD-1 antibody disclosed herein, e.g., nivolumab, pembrolizumab, MEDI0608 (formerly AMP-514), AMP-224, or BGB-A317.

The present invention is further illustrated by the following examples which should not be construed as further limiting. The contents of all references cited throughout this application are expressly incorporated herein by reference.

### Example 1

Study CA008-002 is a Phase I/IIa clinical trial of ADC (I), either as a monotherapy or in combination with nivolumab. The purpose of the trial is to determine the safety, tolerability, pharmacokinetics, immungenecity, antitumor activity, and pharmacodynamics of ADC (I) administered alone and in combination with nivolumab in subjects with mesothelioma, non-small cell lung cancer, ovarian cancer, pancreatic cancer and gastric cancer.

The inclusion/exclusion criteria for assessing subjects for the trial are listed in Table I below.

TABLE I
Inclusion/Exclusion Criteria for Study CA008-002

| Inclusion Criteria | Exclusion Criteria |
|---|---|
| Must have pancreatic, ovarian, | Cancer metastases in the brain |
| gastric, non-small cell lung cancer | |
| or mesothelioma. For dose expansion, | |
| must have tumor that is positive for | |
| mesothelin | |
| Expected to have life expectancy of | Moderate eye disorders |
| at least 3 months | |
| Men and women 18 years old or | Active infection or past hepatitis B |
| older (or local age of majority) | or C infection |
| must have measurable tumor per | Uncontrolled heart disease |
| RECIST or modified RECIST | |
| for malignant pleural mesothelioma | |
| ECOG of 0 or 1 | Impaired liver or bone marrow |
| | function |
| | History of allergy to mesothelin |
| | directed antibodies, tubulysin, |
| | monoclonal antibodies, nivolumab |
| | or related compounds |

The primary end-point is the incidence of adverse events (AEs) at its worst grade, serious adverse events (SAEs) at its worst grade, adverse events leading to discontinuation, deaths, frequency of laboratory test toxicity shifting from baseline, and mean change from baseline of QTc. Safety will be evaluated from the time the subject signs an informed consent and for up to 60 days (100 days for those receiving ADC (I) in combination with nivolumab) after the last dose of study drug.

The secondary end-points include response rate, summary of PK parameters, and anti-drug antibody to ADC (I) alone or in combination with nivolumab. The time frame for monitoring response rate is from day 1 to the last dose of ADC (I) or nivolumab. The time frame for monitoring PK parameters is from day 1 to day 84 for Q3W administration and from day 1 to day 112 for Q1W administration. The time frame for monitoring ADC (I) is from day 1 to day 60 days after the last dose of ADC (I) (100 days for combination therapy with nivolumab).

In Part 3A of the study subjects will be treated with a set dose of nivolumab and increasing doses of ADC (I) until the maximum tolerated dose is reached. The five afore-mentioned cancers will be studied.

Nivolumab will be administered as 360 mg IV Q3W in all dose cohorts, as a 30-minute infusion. The starting dose of ADC (I) to be combined with nivolumab at 360 mg Q3W in Part 3A will be 0.8 mg/kg IV Q3W. In the event that the first dose level of ACD (I) is determined to exceed the maximum tolerated dose (MTD) in combination with 360 mg Q3W of nivolumab, a lower ADC (I) dose such as 0.4 mg/kg and/or 0.2 mg/kg may be explored based on available safety, PK, and biomarker information.

### Example 2

Part 3B of study CA008-002 is a Phase IIa is an expansion of a selected dose of ADC (I). Subjects will be treated at or below the MTD of ADC (I) and a set dose of nivolumab per above.

The purpose of the cohort expansion will be to assess preliminary anti-tumor efficacy, expanded safety experience, and PD effects of ADC (I) in combination with nivolumab. Additional subjects will be treated after the completion of Part 3A, at the MTD or at an alternate dose below the MTD as agreed upon by the Medical Monitor and investigators. Five expansion cohorts will be restricted to these tumor types: 1) mesothelioma, 2) pancreatic, 3) ovarian, 4) NSCLC, and 5) gastric cancer. Enrollment in cohort expansion will be determined by the mesothelin expression of the archived tumor sample (or fresh tumor sample if archived sample is not available). Approximately up to 25 to 26 subjects with an H score ≥100 or with 3+ staining in ≥10% of the cells for tumor mesothelin expression will be treated in each of the 5 cohorts (total 125 to 150 subjects) after sufficient subjects are enrolled from each population to assess preliminary efficacy signal in both populations.

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

What is claimed is:

1. A method of treating a subject afflicted with a tumor comprising administering to the subject:
(a) an antibody or an antigen-binding portion thereof that binds specifically to a Programmed Death-1 (“PD-1”) receptor and inhibits PD-1 activity (“anti-PD-1 antibody”); and
(b) an antibody-drug conjugate of an anti-mesothelin antibody (“anti-mesothelin ADC”).

2. The method of claim 1, wherein the tumor is non-small cell lung cancer (NSCLC), ovarian cancer, mesothelioma, pancreatic cancer, or gastric cancer.

3. The method of claim 2, wherein the tumor comprises one or more cells that express PD-L1 or PD-L2, or both, and one or more cells that express mesothelin.

4. The method of claim 3, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the tumor cells express PD-L1, PD-L2, or both.

5. The method of claim 3, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the tumor cells express mesothelin.

6. The method of any one of claims 1 to 5, wherein the anti-mesothelin antibody in the ADC cross-competes for binding to mesothelin with antibody 6A4.

7. The method of any one of claims 1 to 5, wherein the anti-mesothelin antibody in the ADC comprises antibody 6A4.

8. The method of any of claims 1 to 5, wherein the tubulysin analog has a structure represented by formula (A):

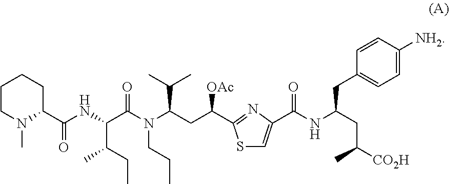 [SMILES: CCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)C[C@H](C)C(=O)O)cs1)C(C)C]

9. The method of any one of claims 1 to 5, wherein the anti-PD-1 antibody cross-competes with nivolumab for binding to human PD-1.

10. The method of any one of claims 1 to 5, wherein the anti-PD-1 antibody comprises a heavy chain constant region which is of a human IgG1 or IgG4 isotype.

11. The method of any one of claims 1 to 5, wherein the anti-PD-1 antibody is nivolumab.

12. The method of any one of claims 1 to 5, wherein the anti-PD-1 antibody is administered at a dose ranging from at least about 0.1 mg/kg to at least about 10.0 mg/kg body weight once about every 1, 2 or 3 weeks.

13. The method of any one of claims 1 to 5, wherein the anti-PD-1 antibody or antigen-binding portion thereof is administered at a flat dose.

14. The method of any one of claims 1 to 5, wherein the anti-PD-1 antibody and anti-mesothelin ADC are administered sequentially.

15. The method of any one of claims 1 to 5, wherein the anti-PD-1 antibody and the anti-mesothelin ADC are administered concurrently in separate compositions.
